(19) United States
(12) Patent Application Publication
(10) Pub. No.: US 2005/0085757 A1
(43) Pub. Date: Apr. 21, 2005

(54) Expandable temporary abdominal closure

(76) Inventor: Steven Santanello, Columbus, OH (US)

Correspondence Address: QUARLES & BRADY LLP, 411 E. WISCONSIN AVENUE, SUITE 2040, MILWAUKEE, WI, 53202-4497, US

(21) Appl. No.: 10/965,525

(22) Filed: Oct. 14, 2004

Related U.S. Application Data
(60) Provisional application No. 60/511,441, filed on Oct. 15, 2003.

(51) Int. Cl. A61F 13/00

(52) U.S. Cl. 602/41

(57) ABSTRACT

A system for temporary abdominal or thoracic closure provides an expanded folded section allowing shielding of the wound from contamination and visual inspection while relieving abdominal pressure.

### CROSS-REFERENCE TO RELATED APPLICATIONS

This application is based on and claims the benefit of U.S. Provisional Application 60/511,441, filed Oct. 15, 2003.

### STATEMENT REGARDING FEDERALLY SPONSORED RESEARCH OR DEVELOPMENT

### BACKGROUND OF THE INVENTION

This invention relates to surgical temporary wound closure devices and the method of using such a device for temporary abdominal and thoracic closure. .

Some abdominal surgery emergencies, especially for trauma and infectious conditions, cannot be solved with a single operation, and multiple re-operations are required.

Trauma, infection, hemorrhage, a ruptured aortic abdominal aneurysm repair, or other catastrophic abdominal events may lead to increased intra-abdominal pressure, which is a life threatening condition if not treated by opening or leaving the abdominal wall open until the increased pressure subsides. Swelling may also accompany some thoracic surgeries which prevents closure immediately following the surgery.

A common surgical practice for leaving an abdomen open is to cover it with a large piece of plastic which is typically cut from an IV or urinary bag. This piece of plastic is sterilized, cut to size, and then sewn to the periphery of the abdominal wall opening and left in place between operations or until the swelling subsides.

One of the characteristics of the IV or urinary bag closure is that it does not expand in response to further increases in intra-abdominal pressure. In order to avoid some of these problems, temporary closure methods allowing repeated fastening and refastening with adjustable separation have been developed. One such device is described in PCT application: WO9009763A1 entitled Wound Closing Method and Device, filed Feb. 26, 1990. Such closure methods require periodic opening of the closure device to inspect the wound and to change the separation of the wound edges to control intra-abdominal pressure.

### BRIEF SUMMARY OF THE INVENTION

The present invention provides a closure system providing a continuous sheet of sterile, biocompatible material which covers an abdominal or thoracic opening. The outer edges of the sheet may be attached to the edges of the wound while the center section of the sheet expands to accommodate increases in pressure or swelling The sheet may include a resealable slit to provide access to the wound as necessary and may be clear to permit observation of the wound through the sheet.

Specifically, the present invention provides an abdominal and thoracic closure system having a sterilizable flexible sheet which is soft and atraumatic to underlying tissues with a peripheral portion attachable to the skin or fascia of the wound; and a central expandable portion that allows for expansion while providing a continuous covering of the wound by the flexible sheet.

Thus it is one object of at least one embodiment of the invention to provide an closure system that controls pressure or swelling by expansion without the need for manual adjustment.

The flexible sheet may be a transparent plastic material.

Thus it is one object of at least one embodiment of the invention to provide a system that allows visual inspection of the wound.

The expandable portion may be at least one fold formed in the central portion of the sterilizable flexible sheet.

Thus it is yet another object of at least one embodiment of the invention to provide a simple method of introducing expandability into materials suitable for wound closure.

The fold may be along a central fold line where opposite sides of the sterilizable flexible sheet at a first layer approach each other, then fold back upon themselves in a second layer extending away from each other for a predetermined distance, and then fold back again upon themselves in a third layer extending again toward each other to meet at the fold line.

Thus it is yet another object of at least one embodiment of the invention to provide a fold that does not intrude into the wound or create pressure on the open portions of the wound.

The three layers may be sealed together at the peripheral portion at ends of the fold line.

Thus it is another object of at least one embodiment of the invention to provide a folding system that nevertheless preserves closure of the wound against contamination.

The seal boundary defined by an inner edge of the sealed three layers may be removed inward from an edge of the sterilizable flexible sheet.

It is a further object of at least one embodiment of the invention to provide an expandable wound closure system that allows trimming the covering to conform to the wound while preserving the sealing of the wound against contamination.

The seal boundary defined by an inner edge of the sealed three layers may be a line perpendicular to the fold line or an angle opening inward about the fold line.

Thus it is another object of at least one embodiment of the invention to provide flexible sealing alternatives accommodating tradeoffs between maximum area of expansion and simplicity of manufacture.

The invention may include an adhesive positioned on the first layer on a side opposite the second and third layers in the peripheral portion.

Thus it is another object of at least one embodiment of the invention to provide a method of securing the closure system to the edges of the wound prior to or in lieu of staples and or sutures.

The sterilizable flexible sheet may include indicia indicating that the third layer is to be placed away from the skin.

It is thus another object of at least one embodiment of the invention to promote correct orientation of the system in emergency conditions.

The invention may include a resealable seam through the third layer allowing opening of the sterilizable flexible sheet after placement on the skin. The resealable seam includes a sealing means selected from the group consisting of: adhesive, adhesive tape, hook and loop fasteners, and zippers.

It is thus another object of at least one embodiment of the invention to provide access to the wound without the need to remove the wound covering.

The invention may include bilateral flexible wings of the same material located on the underside of the first layer, each of which may be tucked under either side of the abdominal or thoracic wall.

It is thus another object of at least one embodiment of the invention to provide an adhesion preventing barrier between the underlying tissue and the abdominal or thoracic wall.

These particular objects and advantages may apply to only some embodiments falling within the claims, and thus do not define the scope of the invention.

### BRIEF DESCRIPTION OF THE DRAWINGS

FIG. 1 is a perspective view of the closure system of the present invention positioned for application above an abdominal wound;

FIG. 2 is a cross-sectional view of the closure system of FIG. 1 taken along lines 2--2 when first applied to the wound;

FIG. 3 is a cross-sectional view similar to that of FIG. 2 showing expansion of the closure system with increased abdominal pressure;

FIG. 4 is a top plan view of the closure system of FIG. 1 showing an area of heat-sealing such as supports an ellipsoidal trimming cut;

FIG. 5 is a fragmentary perspective view of the closure system of FIG. 1 cut along line 2--2 showing an optional opening slit at the center of an expansion fold;

FIG. 6 is a simplified top plan view of the closure system as may be covered by an adhesive surgical drape that does not interfere with the expansion capabilities of the closure system; and

FIG. 7 is a perspective view of an alternative embodiment of the invention providing flexible lateral wings attached to the underside of an upper layer.

### DETAILED DESCRIPTION OF THE PREFERRED EMBODIMENT

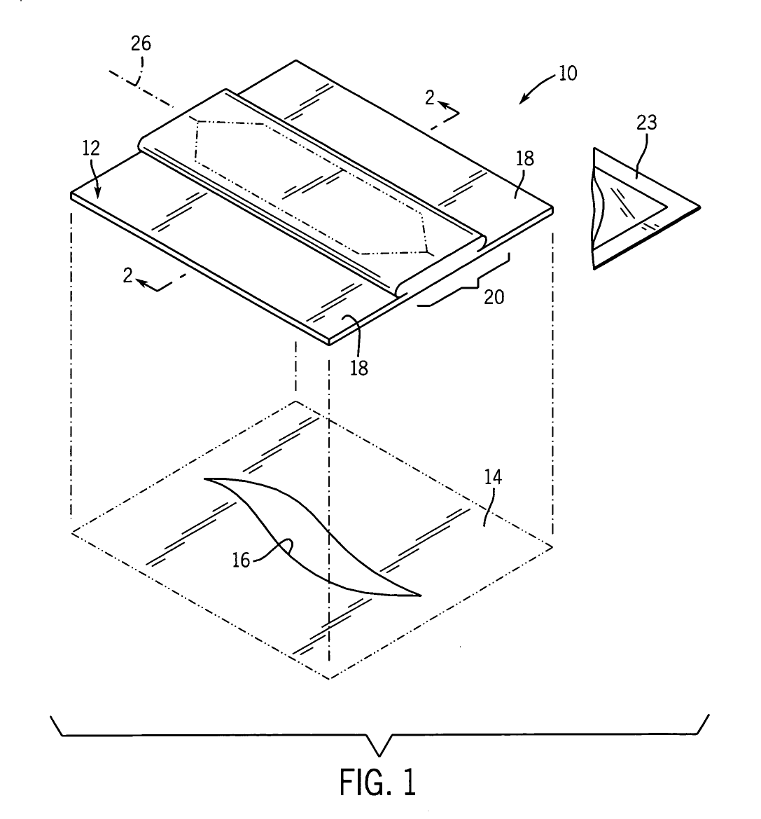

Referring now to FIG. 1, a closure system 10 of the present invention includes a sterile, supple sheet 12 which may, for example, have a rectangular shape and may be formed out of a single sheet of sterilizable biocompatible clear plastic such as polyvinyl chloride (PVC) or other similar materials preferably providing a surface that resists adhesion to tissue.

The closure system 10 is generally applicable to both abdominal and thoracic wounds, but will be described hereafter in the context of an abdominal wound for clarity. Use of the closure system 10 on thoracic wounds will be understood to those of ordinary skill in the art from this description.

The sheet 12 is generally sized to amply cover the skin 14 surrounding a typical abdominal wound 16 so that peripheral portions 18 of the sheet may attach directly to the skin 14 around the wound 16 and a center expandable portion 20 may be positioned above the wound 16. Multiple sizes may be provided with an adult size in one embodiment being 40 cm by 25 cm. The closure system 10 may be provided sealed in a disposable and sterilizable envelope 23 which may be removed prior to application of the sheet 12 to the skin 14 about the wound 16. A second disposable envelope (not shown) may package envelope 23, this envelope removed prior to introduction of envelope 23 into the sterile surgical field.

As an alternate to skin attachment both sides of sheet 12 may be slid through the opening of the wound 16 under the abdominal wall and both sides advanced laterally. The center expandable portion 20 is positioned in the center of the opening of the wound 16.

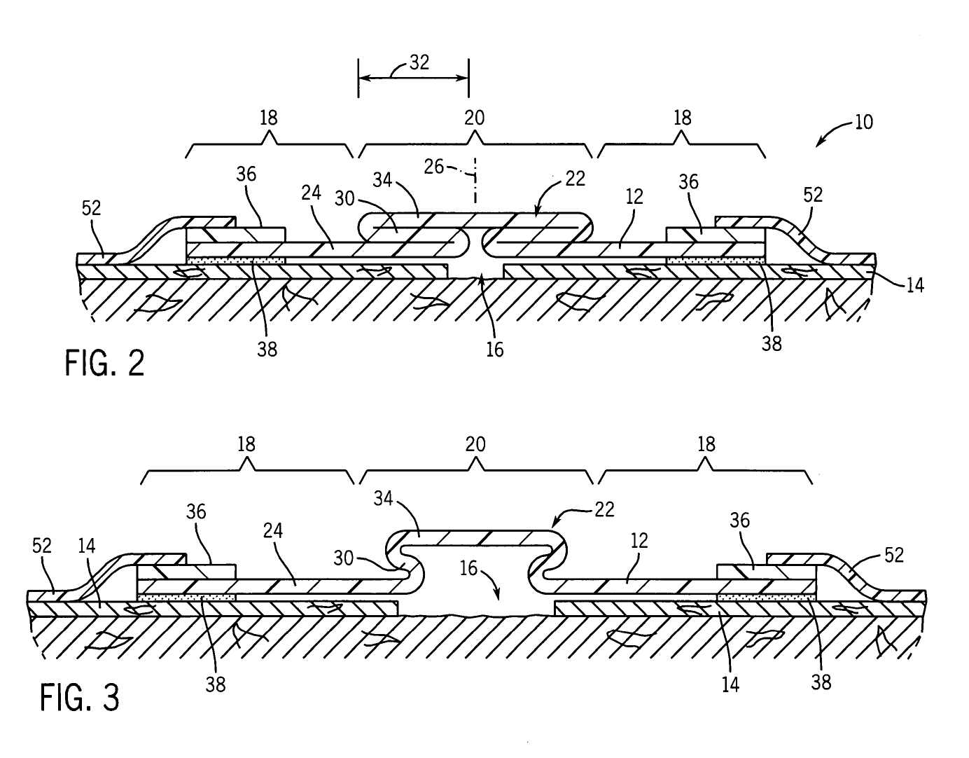

Referring now to FIG. 2, the expandable portion of the sheet 12 is provided with expandability via a fold 22 in sheet 12 extending along a fold line 26 separating laterally opposed peripheral portions 18. The fold 22 allows relatively free expansion of the wound 16, in contrast to an elastic member, while the peripheral portions 18 relieve stress from the superior and inferior ends of the wound 16.

Specifically, the sheet 12 extends inward from the laterally opposed peripheral portions 18 toward the fold line 26 at a first layer 24. At the fold line 26 these two portions of the sheet 12 folds back upon themselves in a second layer 30 now extending away from the fold line 26 for a predetermined fold distance 32. When these portions of the sheet 12 at the second layer 30 reach the predetermined fold distance 32 away from the fold line 26, they fold back again inward upon themselves to form a third layer 34 extending toward each other again to meet at the fold line 26.

Referring now also to FIG. 3, it will be understood that the fold 22 allows the peripheral portions 18 to separate along a lateral direction perpendicular to the fold line 26 expanding the expandable portion 20. This expansion allows the relief of increased abdominal pressure and can be detected visually by observing an unfolding of the fold 22 which causes the third layer 30 to move away from the wound 16.

Referring still to FIGS. 2 and 3, the peripheral portions 18 of the sheet 12 may optionally be reinforced with reinforcement strips 36 adhered to an upper surface or lower surface of the peripheral portions 18 through adhesive or thermal bonding. A pressure sensitive adhesive 38 may be placed on the lower surface of the sheet 12 at the peripheral portions 18 to assist in locating and holding those portions against the skin 14 either independently or in conjunction with sutures or staples. The adhesive 38 may be covered with a protective release liner (not shown) removed prior to use.

The fold 22 extends upward away from the skin 14 so that the layers in order, moving away from the skin 14, are the first layer 24, the second layer 30, and the third layer 34.

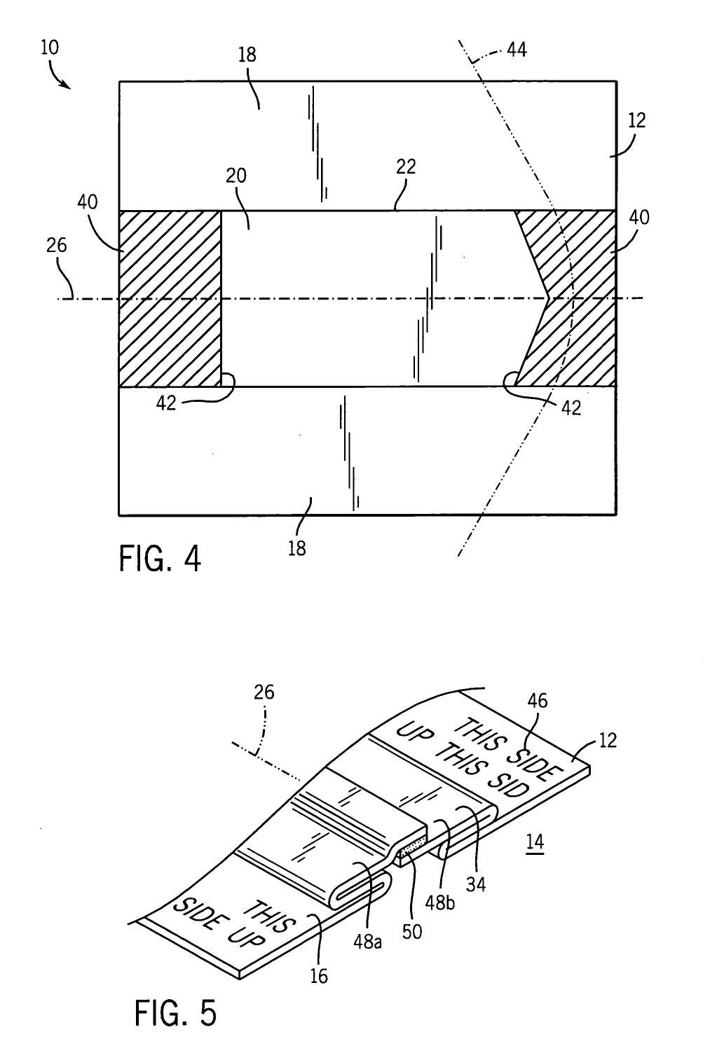

Referring now to FIG. 4, ends of the fold 22 along fold line 26 may be heat sealed to create a heat seal area 40 in opposed superior-inferior peripheral portions 18 of the sheet 12. The heat-sealing joins the layers 24, 30, and 34 in the heat seal area 40. An inner edge 42 of the heat seal area 40 is displaced from the superior-inferior edges of the sheet 12 to allow a trimming cut 44 to be made without breaking the seal established by the seal areas 40. The inner edge 42 may be an angular edge about trimming cut 44 opening inward to conform generally to an expected ellipsoidal trimming cut 44 or may be straight edge for manufacturing simplicity.

Referring now to FIG. 5, an upper surface of the peripheral portions 18 may be printed with indicia 46 indicating that the proper side of the sheet 12 is upward when the sheet 12 is so applied.

A center of the third layer 34 may be slit to produce two separable portions 48a and 48b near the center of the closure system 10, for example, along the fold line 26 that may be opened for inspection or cleaning of the wound and then closed again and sealed either by a sealing means 50 such as hook and loop fasteners, adhesive tape, or a zipper, or similar zipper-like or closure structure. Sealing tape (not shown) may be provided to further seal zipper or hook and loop sealing means.

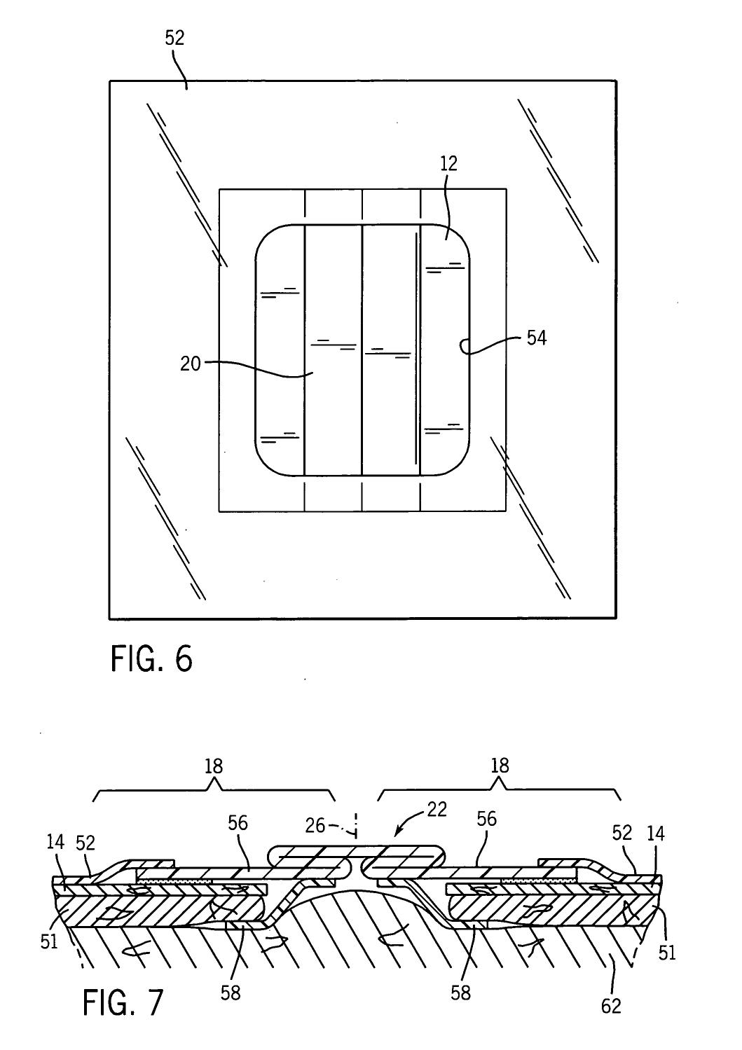

Referring now to FIG. 6, the sheet 12 once installed on the patient may be covered by an adhesive surgical drape 52 having a center cutout 54 suitable to expose the expandable portion 20 thereby not to interfere with its expansion. This seal with the adhesive surgical drape 52 can be used to further stabilize the closure system 10, prevent introduction of contaminants, and create an air tight seal. Alternately, strips of adhesive surgical drape can be cut and fixed to the periphery of the closure system 10 to further stabilize it, prevent introduction of contaminants, and create an air tight seal. Referring now to FIG. 7, in an alternative embodiment, the peripheral portions 18 of the sheet 12 may be constructed of an upper layer 56 and a lower layer 58, the latter attached at an inner edge to the lower surface of the upper layer 56 so that the lower layer 58 extends outward away from the fold line and so that the lower layer 58 may fit between the abdominal wall 51 and the tissue of the abdominal cavity 62 while the upper layer 56 may lie on top of the skin 14 to be attached thereto as described above.

In an alternative embodiment, not shown, the lower layer 58 may be eliminated and the upper layer 56 placed between the abdominal wall 51 and tissue of the abdominal cavity 62.

It is specifically intended that the present invention not be limited to the embodiments and illustrations contained herein, but include modified forms of those embodiments including portions of the embodiments and combinations of elements of different embodiments as come within the scope of the following claims.

1. An abdominal and thoracic closure system comprising:
a sterilizable flexible sheet comprising:
a peripheral portion attachable to skin or fascia about a wound; and
a central expandable portion allowing expansion of the abdomen or thorax while providing a continuous covering of a wound in the abdomen by the flexible sheet.

2. The closure system of claim 1 wherein the flexible sheet is a transparent plastic material.

3. The closure system of claim 1 wherein the expandable portion is at least one fold formed in the central portion of the sterilizable flexible sheet.

4. The closure system of claim 3 wherein the fold is along a central fold line where opposite sides of the sterilizable flexible sheet at a first layer approach each other, and then fold back upon themselves in a second layer extending away from each other for a predetermined distance, and then fold back again upon themselves in a third layer extending again toward each other to meet at the fold line.

5. The closure system of claim 4 wherein the three layers are sealed together at the peripheral portion at ends of the fold line.

6. The closure system of claim 5 wherein a seal boundary, defined by an inner edge of the sealed three layers, is removed inward from an edge of the sterilizable flexible sheet.

7. The closure system of claim 5 wherein a seal boundary, defined by an inner edge of the sealed three layers, is a line perpendicular to the fold line.

8. The closure system of claim 5 wherein a seal boundary defined by an inner edge of the sealed three layers is an angle opening inward about the fold line.

9. The closure system of claim 4 including an adhesive positioned on the first layer on a side opposite the second and third layers in the peripheral portion.

10. The closure system of claim 4 further including indicia on the sterilizable flexible sheet indicating that the third layer is to be placed away from the skin.

11. The closure system of claim 4 further including a resealable seam through the third layer allowing opening of the sterilizable flexible sheet after placement on the skin.

12. The closure system of claim 11 wherein the resealable seam includes a sealing means selected from the group consisting of: adhesive, adhesive tape, hook and loop fasteners, and zippers.

13. The closure system of claim 11 wherein the flexible sheet is a transparent plastic material.

14. The closure system of claim 1 wherein the peripheral portion includes a downwardly facing adhesive for holding the peripheral portion against the skin.

15. The closure system of claim 1 further including printed indicia on the flexible sheet indicating a side to be placed against the skin.

16. The closure system of claim 1 further including a sterilizable disposable pouch for holding the sterilizable flexible sheet against contamination prior to use.

17. The closure system of claim 1 wherein the peripheral portions provide an upper and lower layer separable so that the upper layer may lay against the outer surface of the skin and the lower layer may fit between a cavity wall and an underlying cavity.

18. A method of treating abdominal and thoracic wounds using a closure system of a type providing a sterilizable flexible sheet having a peripheral portion, a central expandable portion allowing expansion of the central portion without substantial expansion of the peripheral portion comprising the steps of:
applying the peripheral portion to skin surrounding the wound with the central expandable portion aligned with the wound; and
applying an adhesive surgical dressing to the peripheral portion to attach the peripheral portion to the skin, the adhesive surgical dressing exposing the central expandable portion to allow expansion of that portion.
